(19) United States
(12) Patent Application Publication
(10) Pub. No.: US 2005/0133762 A1
(43) Pub. Date: Jun. 23, 2005

(54) Liquid crystalline compound having high optical anisotropy and liquid crystalline composition comprising the same

(75) Inventors: Eun Kyung Lee, Seoul (KR); Ji Young Jong, Seoul (KR); Hyung Soo Cho, Daegu-si (KR); Byung Soon Chen, Gyeonggi-Do (KR); Yoon Sok Kang, Gyeonggi-Do (KR)

Correspondence Address: BIRCH STEWART KOLASCH & BIRCH, PO BOX 747, FALLS CHURCH, VA, 22040-0747, US

(73) Assignee: Samsung Electronic Co., Ltd., Suwon-si (KR)

(21) Appl. No.: 10/926,021

(22) Filed: Aug. 26, 2004

(30) Foreign Application Priority Data
Nov. 28, 2003 (KR) 2003-85508

(51) Int. Cl. C09K 19/32; C07C 25/00

(52) U.S. Cl. 252/299.62; 428/1.1; 570/183

(57) ABSTRACT

A novel liquid crystalline compound having a negative dielectric anisotropy and a high optical anisotropy. Since the rotation viscosity and the elasticity coefficient (K11/K33) of the liquid crystalline compound are maintained at a low level, the liquid crystalline compound can be effectively used as a liquid crystalline medium having good image quality and high response speed even when applied to thin liquid crystalline cells.

### BACKGROUND OF THE INVENTION

This non-provisional application claims priority under 35 U.S.C. § 119(a) on Korean Patent Application No. 2003-85508 filed on Nov. 28, 2003, which is herein incorporated by reference.

1. Field of the Invention

The present invention relates to a novel liquid crystalline compound and a liquid crystalline composition comprising the same, and more particularly to a liquid crystalline compound having a negative dielectric anisotropy and a high optical anisotropy, and a liquid crystalline composition comprising the liquid crystalline compound.

2. Description of the Related Art

A variety of flat panel displays, including liquid crystal displays (LCDs), field emission displays (FEDs), plasma display panels (PDPs) and electroluminescent display (ELDs), are very slim and lightweight, and can be fabricated to have a large area. Based on these advantages, flat panel displays are increasingly used in a wide variety of applications, such as notebook monitors and display devices for use in aircraft cockpit compartments, medical devices, navigational instruments, measuring instruments, and the like.

Liquid crystalline displays (LCDs) have the highest market share of the flat panel display products because of their easy portability and low power consumption. Liquid crystalline displays can be classified into projection type LCDs and direct view type LCDs. The direct view type LCDs are devices where a viewer can directly view light generated from the LCDs, and they are sub-classified into transmissive and reflective LCDs. The former is a device wherein the intensity of light produced from a backlight is regulated by an LCD panel, and the latter is a device wherein natural light and ambient light are reflected from the LCD panel to form desired images. In particular, a noticeable display device, LCOS (Liquid Crystal on Silicon) microdisplay comprises a silicon back plate and a cover glass, both of which are conductors and have mirror-like surfaces as a pixel array, and a liquid crystalline material is introduced between the two components. Although the LCOS microdisplay has a diagonal length of 1 inch or below, high-resolution images can be obtained. Since the LCOS microdisplay generally has small-sized pixels, it is composed of about 1 μm thick thin cells. In view of d·Δn values, liquid crystalline media used in the LCOS microdisplay are required to have a high optical anisotropy of more than 0.1, unlike a general transmissive liquid crystalline display having an optical anisotropy (i.e., refractive index anisotropy (Δn)) of 0.1 or less.

Liquid crystalline compounds applied to a vertical alignment (VA) technology should exhibit a relatively high negative dielectric anisotropy. In addition, these liquid crystalline compounds for VA are required to have the following properties: (1) a particular optical anisotropy in their liquid crystalline phase; (2) a low K33/K11 ratio and a low rotation viscosity so as to ensure a high response speed; (3) a chemical stability against external factors such as UV rays, heat, infrared rays, air, electric fields, etc.; and a liquid crystalline phase over a broad temperature range.

There has been no report regarding a single liquid crystalline compound simultaneously having a high optical anisotropy and a high negative dielectric anisotropy sufficient to be fabricated into VA mode thin liquid crystalline cells, until now. Accordingly, liquid crystalline compositions comprising about 5 to 25 liquid crystalline compounds are currently used to exhibit intended liquid crystalline properties, but they fail to satisfy the above-mentioned requirements for an ideal VA liquid crystalline composition. Thus, there is a need for a novel liquid crystalline compound having a high optical anisotropy, a high negative dielectric anisotropy, a low rotation viscosity and a broad liquid crystalline temperature range.

### SUMMARY OF THE INVENTION

According to the present invention it has been found that a liquid crystalline compound containing a particular naphthalene moiety has a negative dielectric anisotropy, a high optical anisotropy, a low rotation viscosity, a low K33/K11 ratio and a broad liquid crystalline temperature range, thus accomplishing the present invention.

Therefore, a feature of the present invention is to provide a novel liquid crystalline compound which can be applied to VA mode thin liquid crystalline cells due to its negative dielectric anisotropy and high optical anisotropy, and has a high response speed while ensuring good image quality.

In accordance with a feature of the present invention, there is provided a liquid crystalline compound represented by Formula 1 below: 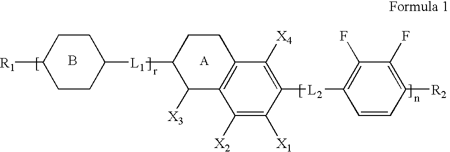
wherein R₁ and R₂ are each independently C₁₋₂₀ alkyl, cycloalkyl, alkoxy or alkenyl group in which 1-4 of the hydrogen atoms may be substituted with fluorine atoms; X₁, X₂, X₃ and X₄ are each independently a hydrogen atom, F, Cl, Br, NCS, CN, CH₃, CF₃, CHF₂, CH₂F, OCF₃, OCHF₂ or OCH₂F; L₁ and L₂ are each independently a single bond, a C₁-4 alkylene group, a C₂-4 divalent unsaturated hydrocarbon group containing at least one double or triple bond, —COO—, —OCO—, —CH₂O—, —CF₂O—, —OCF₂—, —OCH₂—, —NHCH₂—, —CH₂NH—, —CH₂CO—, —COCH₂—, —N═N— or —NON—; n is an integer of 1 or 2; r is an integer of 0 to 2; 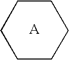
   constituting the naphthalene group is a cyclohexane, cyclohexene or a benzene ring; 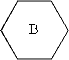
   is 1,4-cyclohexylene, 1,4-phenylene or a cyclohexene-1,4-diyl group in which at least one hydrogen atom may be substituted with a fluorine atom.

In accordance with another feature of the present invention, there is provided a liquid crystalline composition comprising the liquid crystalline compound of Formula 1.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is ¹H NMR data of the liquid crystalline compound prepared in Synthetic Example 1; and

FIG. 2 is ¹H NMR data of the liquid crystalline compound prepared in Synthetic Example 2.

### DETAILED DESCRIPTION OF THE PREFERRED EMBODIMENT

Hereinafter, the present invention will be explained in more detail.

Since the liquid crystalline compound of Formula 1 contains a naphthalene or hydrogenated naphthalene moiety as a rigid ring moiety, and a 1,4-phenylene group substituted with two fluorine atoms attached to one side of the naphthalene moiety, it shows a high optical anisotropy and a high negative dielectric anisotropy. Where at least one electron-withdrawing group, such as F, Cl, Br, NCS, CN and OCF₃, is substituted to the naphthalene moiety of the liquid crystalline compound, the negative dielectric anisotropy of the liquid crystalline compound become higher.

Preferred embodiments of the crystalline compound according to the present invention are those wherein R₁ is C₃₋₁₀ alkyl group, R₂ is C₂₋₁₀ alkyl, alkoxy or cyclohexyl group in which 1-4 of hydrogen atoms may be substituted with fluorine atoms; X₁, X₂, X₃ and X₄ are each independently a hydrogen atom, F, Cl, CH₃, CF₃, CHF₂, CH₂F, OCF₃, OCHF₂ or OCH₂F; L₁ and L₂ are each independently a single bond, methylene, ethylene, —CH═CH—, —C≡C—, —CH₂O—, —CF₂O—, —OCH₂—, —NHCH₂—, —CH₂NH— or —COCH₂—; n is 1; and r is 0 or 1.

More preferred compounds according to the present invention are those represented by Formulae 2 to 4 below:

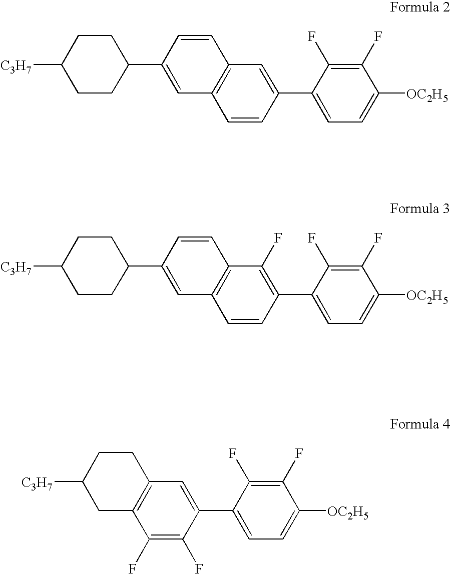

The compounds of the present invention can be prepared through appropriate synthetic paths, for instance, in accordance with Reaction Scheme 1 or 2 below.

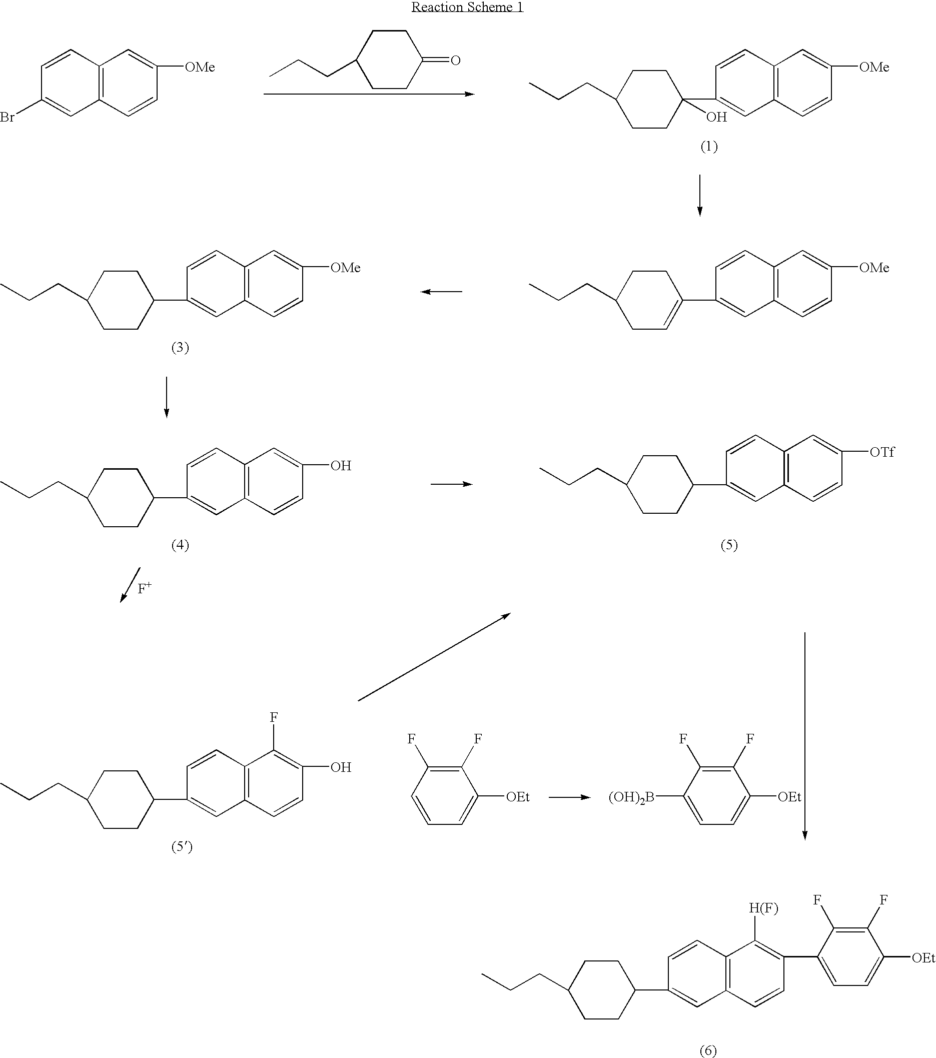

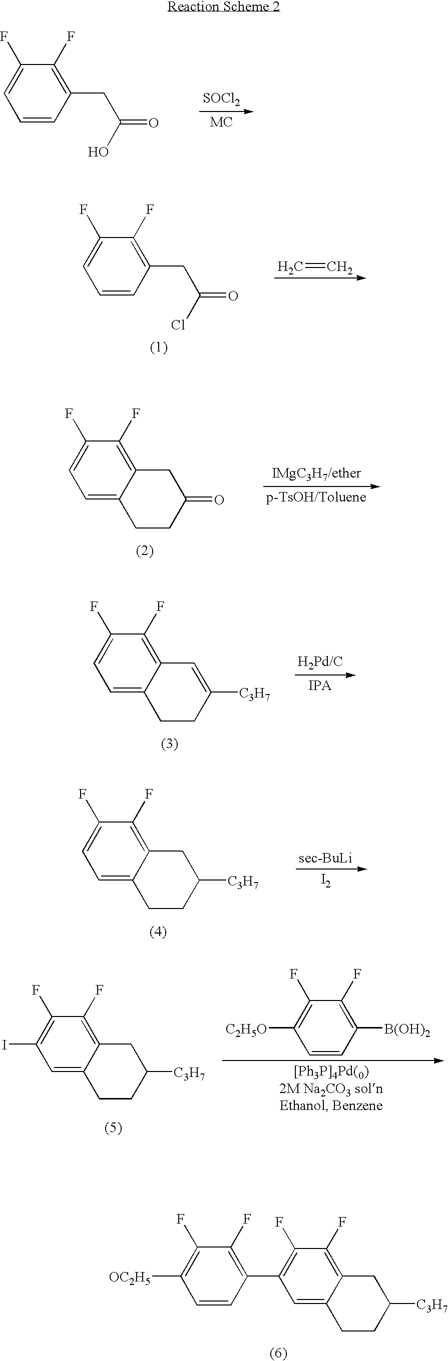

The present invention also provides a liquid crystalline composition comprising the compound of Formula 1. The liquid crystalline compounds of Formula 1 may be used alone or in combination. Alternatively, a previously known liquid crystalline compound may be further added in order to appropriately control the physical properties and various optical parameters of the liquid crystalline composition. Examples of such known liquid crystalline compounds include, but are not limited to, liquid crystalline compounds containing a cyclohexylphenyl group for viscosity reduction. Specific examples of known liquid crystalline compounds are as follows (see, e.g., V. Reiffenrath et al., Liq. Cryst., 5 (1) 159 (1989): M. Klasen-Memmer et. al., IDW (international display workshop) 2002, 93 (Hiroshima, Japan): M. Heckmeier et al., U.S. Pat. No. 6,515,580 (2003): K. Miyazawa et al., U.S. Pat. No. 6,348,244(2002)). 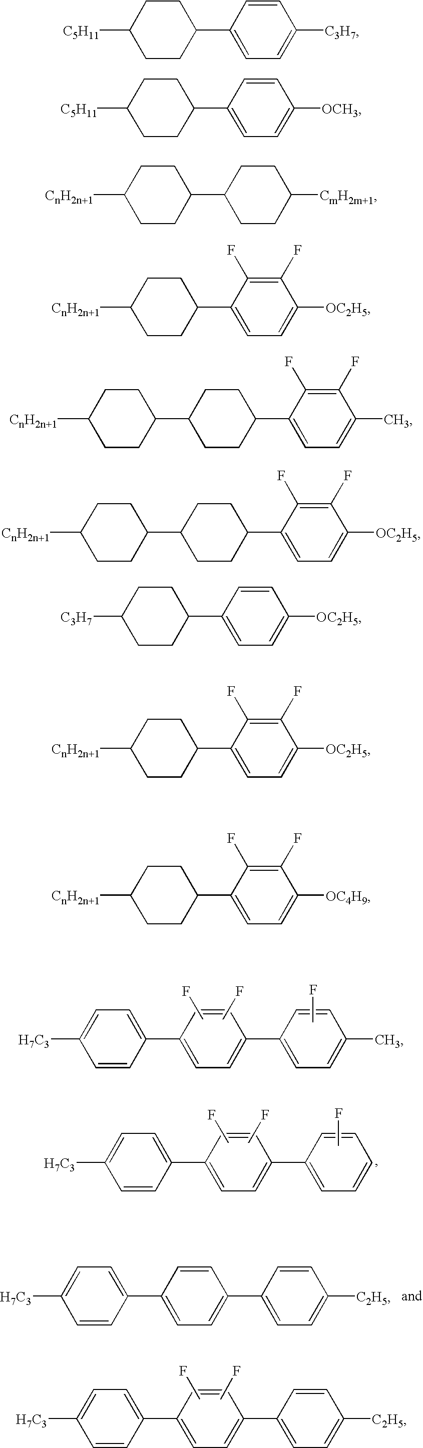
wherein n is an integer of from 1 to 10 and n+m is in the range of 3˜15, but n and m are not specifically limited to these ranges.

The content of the liquid crystalline compound according to the present invention in the liquid crystalline composition is not especially limited, but is preferably in the range of 3˜50% by weight, based on the total weight of the composition. The liquid crystalline composition of the present invention has preferably an optical anisotropy as high as 0.05˜0.3, and more preferably 0.15˜0.25. The liquid crystalline composition of the present invention has an absolute value of 2 or more (i.e., −2.0 or less), more preferably 3 or more in negative dielectric anisotropy and is appropriate for the use in VA mode thin liquid crystalline cells.

Hereinafter, the present invention will be described in more detail with reference to the following preferred examples. However, these examples are given for the purpose of illustration and are not to be construed as limiting the scope of the invention.

### SYNTHETIC EXAMPLE 1

The compounds of Formulae 2 and 3 were prepared in accordance with the above Reaction Scheme 1.

1) Preparation of 2-methoxy-6-(1-hydroxy-4-propylcyclohexyl)naphthalene (1)

30 g (0.126 mol) of 2-bromo-6-methoxynapthalene is dissolved in 300 ml of THF, and then cooled to −78° C. To the solution is added 60 ml (0.163 mol) of n-BuLi. The resulting mixture is stirred for 4 hours. Then, 14.7 g (0.105 mol) of n-propylcyclohexanone is added to the mixture and the reaction temperature is raised to room temperature followed by stirring the mixture. The reaction is completed by the addition of 5% HCl solution. The resulting compound is extracted with ether, dried over MgSO₄ and filtered. The solvent is evaporated to yield the target compound as a yellow solid (yield: 80%).

GC mass data: m/z 298 (80%) 2) Preparation of 2-methoxy-6-(4-propylcyclohexenyl)naphthalene (2)

40 g of the crude product of 2-methoxy-6-(1-hydroxy-4-propyl) cyclohexylnaphthalene prepared in step 1) is dissolved in 300 ml of toluene, and 0.5 g of p-TsOH is added thereto. The resulting mixture is refluxed for 4 hours and the reaction is completed by the addition of a saturated NaHCO₃ solution. The resulting compound is extracted with toluene, dried over MgSO₄ and filtered. The solvent is evaporated to yield the target compound as a white solid (yield: 95%).

GC mass data: m/z 280 (>99%)

3) Preparation of 2-methoxy-6-(4-propylcyclohexyl)naphthalene (3)

21 g (0.075 mol) of 2-methoxy-6-(4-propylcyclohexenyl)naphthalene prepared in step 2) is dissolved in a mixture of 300 ml of toluene and 250 ml of ethanol 250 ml, and then 4 g of Pd/C is added thereto. Hydrogenation is conducted in the presence of 5 bar of H₂ at 40° C. After 24 hours, the reaction mixture is passed through Celite to remove the catalyst Pd/C, and then the solvents are removed. The condensate is recrystallized in toluene and hexane to yield the target compound as a white solid (yield 65%)

GC mass data: m/z 282 (>99%)

4) Preparation of 2-(4-propylcyclohexyl)naphth-6-ol (4)

To 20 g (0.07 mol) of 2-methoxy-6-(4-propylcyclohexyl)naphthalene prepared in step 3) is added 16.5 g (0.14 mol) of pyridine hydrochloride. The resulting mixture is refluxed at 200° C. The resulting compound is extracted with ether, washed with water three times or more, dried over MgSO₄ and filtered. The solvent is evaporated to yield the target compound as a white solid (yield: 70%).

GC mass data: m/z 268 (>99%)

Meanwhile, in order to prepare the compound of Formula 3 in which one fluorine atom is substituted to the naphthalene moiety, 1-fluoro-6-(4-propylcyclohexyl)naphth-2-ol (5′) is prepared as follows:

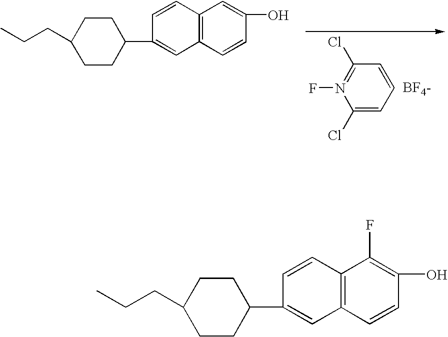

Specifically, 2 g (0.007 mol) of 2-(4-propylcyclohexyl)naphth-6-ol (4) is dissolved in 70 ml of dichloromethane under nitrogen atmosphere, and then 2.3 g (0.009 mol) of a fluorination agent is added thereto. The resulting mixture is stirred for one day and the reaction is completed by the addition of water. The resulting compound is extracted with dichloromethane, washed with water three times, dried over MgSO₄ and filtered. The solvent is evaporated to yield the title compound as a red liquid (yield: 90%).

GC mass data: m/z 286 (>99%)

5) Preparation of 2-(4-propylcyclohexyl)naphth-6-oxytriflate (5)

2 g (0.007 mol) of 2-(4-propylcyclohexyl)naphth-6-ol prepared in step 4) was dissolved in 15 ml of pyridine, and then 4.2 g (0.014 mol) of (OTf)₂O is slowly added thereto. The resulting mixture is stirred at room temperature for 3 hours and the reaction is completed by the addition of water. The resulting compound is extracted with ether, washed with water three times or more, dried over MgSO₄ and filtered. The solvent is evaporated to yield the target compound as a yellow liquid (yield: 70%)

GC mass data: m/z 401 (>99%)

On the other hand, 1-Fluoro-6-(4-propylcyclohexyl)naphth-2-ol (5′) prepared in step 4) is treated in the same manner as the above procedure to prepare 1-fluoro-6-(4-propylcyclohexyl)naphth-2-oxytriflate.

6) Preparation of 2-(4-propylcyclohexyl)-6-{2,3-difluoro-4-ethoxybenzyl}naphthalene (6) and fluorinated compound thereof

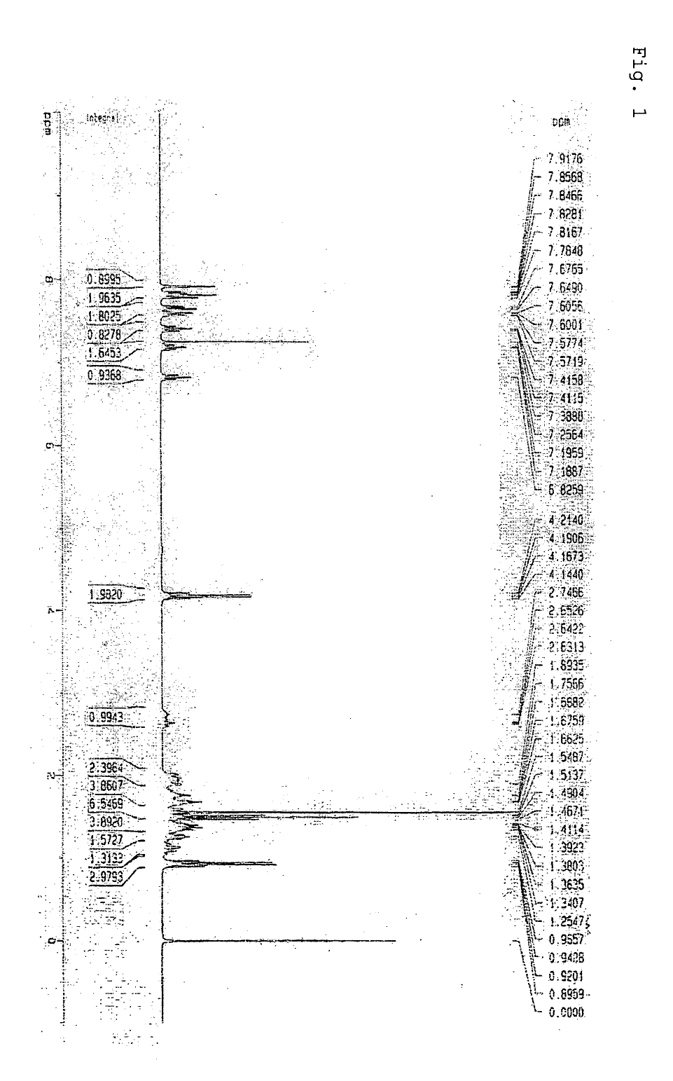

8 g (0.02 mol) of 2-(4-propylcyclohexyl)naphth-6-oxytriflate or 1-fluoro-6-(4-propylcyclohexyl)naphth-2-oxytriflate prepared in step 5) is dissolved in 30 ml of benzene, and then 0.5 g of tetrakis(triphenylphosphine)Pd(0) is added thereto. 25 ml of 2M Na₂CO₃ was added to the resulting mixture, and then a solution of 4.43 g (0.021 mol) of 2,3-difluoro-4-ethoxybenzylboronic acid in 50 ml of ethanol is added thereto. The resulting mixture is refluxed at 100° C. for one day and the reaction is completed by the addition of water. The resulting compound is extracted with toluene, washed with water three times or more, dried over MgSO₄ and filtered. The solvent is evaporated followed by separation using column chromatography (hexane) to yield the target compound as a white solid (yield: 70%). FIG. 1 shows ¹H NMR data of the obtained 2-(4-propylcyclohexyl)-6-{2,3-difluoro-4-ethoxybenzyl}naphthalene. The molecular mass of the compound is determined by GC mass, showing the following result:

GC mass data: m/z 408 (>99%)

### SYNTHETIC EXAMPLE 2

The compound of Formula 4 is prepared in accordance with the above Reaction Scheme 2.

1) Preparation of (2,3-difluorophenyl)acetyl chloride (1)

1.0 mole of (2,3-difluorophenyl)acetic acid is dissolved in dichloromethane and 1.1 mole of thionyl chloride is added. The mixture is heated to 35° C. and stirred for 4 hours. After completion of the reaction, water is added. The resulting compound is extracted with dichloromethane, dried over MgSO₄, and filtered. The solvent is evaporated to yield the target compound. (yield 80%)

GC mass data: m/z 193 (>99%)

2) Preparation of 7,8-difluoro-3,4-dihydro-1H-naphthalen-2-one (2)

1.0 mole of (2,3-difluorophenyl)acetyl chloride is dissolved in isopropyl alcohol and 0.1 mole of 5% Pd/C is added. To the mixture, is added ethylene gas dropwise and stirred at 40° C. for 12 hours. The reactant is filtered through Celite and the solvent is evaporated to yield the target compound. (yield 70%)

GC mass data: m/z 182 (>99%)

3) Preparation of 5,6-difluoro-3-propyl-1,2-dihydronaphthalene (3)

1.3 mole of magnesium is dissolved in anhydrous ether and 1.5 mole of iodopropane is slowly added dropwise. To the mixture, is dropped a solution of 1.0 mole of 7,8-difluoro-3,4-dihydro-1H-naphthalen-2-one in ether. The mixture is stirred for 4-5 hours and water is added to complete the reaction. The resulting compound is extracted with ether, dried over MgSO₄ and filtered. The solvent is evaporated to yield the target compound. (yield 75%)

GC mass data: m/z 180 (>99%)

4) Preparation of 7,8-difluoro-2-propyl-1,2,3,4-tetrahydronaphthalene (4)

1.0 mole of 5,6-difluoro-3-propyl-1,2-dihydronaphthalene mole is dissolved in ethanol and 0.1 mole of Pd/C is slowly added. The mixture is stirred for 4 hours at 40° C. under 5 bar of H₂ pressure. The reactant is filtered through Celite and the solvent is evaporated to yield the target compound. (yield 82%)

GC mass data: m/z 182 (>99%)

5) Preparation of 7,8-difluoro-6-iodo-2-propyl-1,2,3,4-tetrahydronaphthalene (5)

1.0 mole of 7,8-Difluoro-2-propyl-1,2,3,4-tetrahydronaphthalene is dissolved in THF and cooled to −78° C. 1.5 mole of BuLi is added and stirred for 4 hours. Then, after 1.5 mole of iodine is added, the temperature is raised to room temperature and reaction is continued for 4 hours. 10% HCl solution is added to complete the reaction. The resulting compound is extracted with ether, dried over MgSO₄ and filtered. The solvent is evaporated to yield the target compound. (yield 60%)

GC mass data: m/z 261 (>99%)

6) Preparation of 6-(4-ethoxy-2,3-difluorophenyl)-7,8-difluoro-2-propyl-1,2,3,4-tetrahydronaphthalene (6)

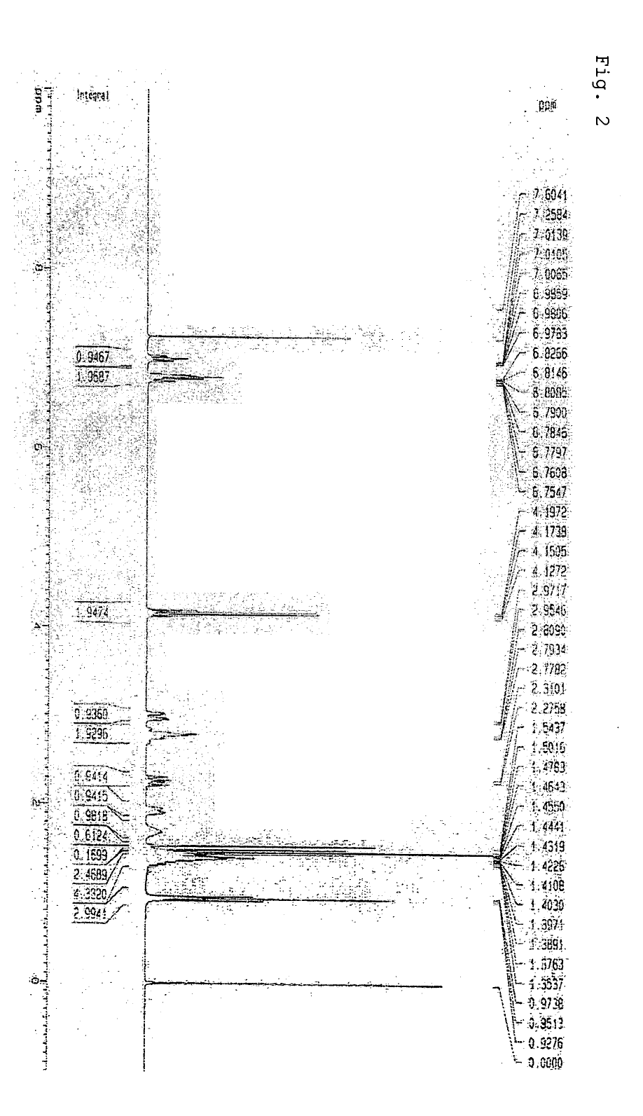

1.0 mole of 7,8-difluoro-6-iodo-2-propyl-1,2,3,4-tetrahydronaphthalene is dissolved in benzene and 0.1 mole of tetrakis(triphenylphosphine)Pd(0) is added. 1.0 mole of 2M Na₂CO₃ solution is added further. Separately, 1.1 mole of 1-ethoxy-2,3-difluorophenyl boronic acid is dissolved in ethanol and the solution is added to the above mixture. The reaction mixture is refluxed for 24 hours and water is added to complete the reaction. The resulting compound is extracted with toluene, dried over MgSO₄ and filtered. The solvent is evaporated to yield the target compound. (yield 76%) FIG. 2 shows ¹H NMR data of the obtained 6-(4-ethoxy-2,3-difluorophenyl)-7,8-difluoro-2-propyl-1,2,3,4-tetrahydronaphthalene. The molecular mass of the compound is determined by GC mass, showing the following result:

GC mass data: m/z 338 (>99%)

Evaluation of Liquid Crystalline Properties

Transition temperature of the liquid crystalline compounds prepared in Synthetic Examples are measured and shown in Table 1 below.

TABLE 1

| Liquid Crystalline Compound | Transition Temperature |
|---|---|
| 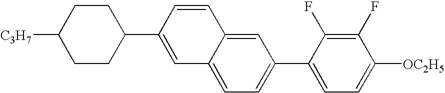 | Cr 128 N 228 I |
| 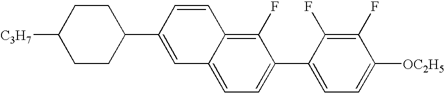 | Cr 151 N 218 I |
| 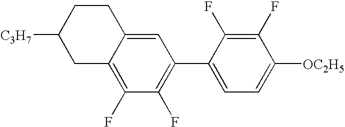 | Cr 83 i |

In addition, Liquid crystalline compositions comprising the liquid crystalline compounds prepared in the Synthetic Examples are prepared to have the compositions as indicated in Table 2 below. The optical anisotropy (Δn), Tni and dielectric anisotropy (Δε) of the respective liquid crystalline compositions are measured. Specifically, the respective liquid crystalline properties are measured in accordance with the following procedures. The liquid crystalline composition is injected into a vertically aligned liquid crystalline cell, and the dielectric anisotropy (Δε) is measured using a measurement system (Model 6254, Toyo Company) at 20° C. and 0.1 Hz. The optical anisotropy (Δn) is obtained by measuring the refractive index to normal light and abnormal light at 20° C. using an interference filter of an Abbe refractometer (589 nm). Parameters in connection with the liquid crystalline properties are as follows:
Δn: Optical anisotropy vale at 20° C. (measured at 589 nm)
TNI (° C.): Nematic anisotropy transition temperature

Δε: Dielectric anisotropy at 20° C. (measured at 0.1 Hz)

TABLE 2

| | Content (wt %) | | | | | |
|---|---|---|---|---|---|---|
| Liquid Crystalline Compound | A | B | C | D | E | F |
| 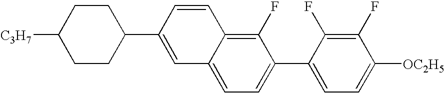 | 5 | | | | | |
| 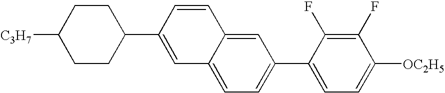 | | 10 | | 9 | 12 | 16 |
| 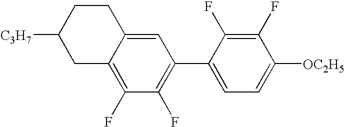 | | | 10 | | 10 | 5 |
| 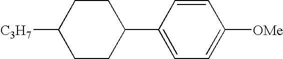 | 3 | 3 | 2 | 2 | 2 | 2 |
| 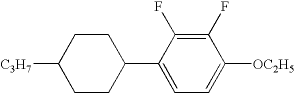 | 40 | 38 | 3 | 3 | 3 | 3 |
| 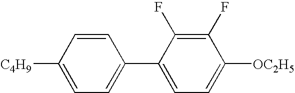 | 9 | 8 | | | | |
| 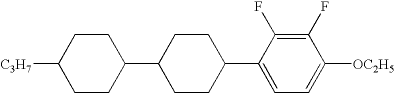 | 9 | 9 | 8 | 8 | 6 | 6 |
| 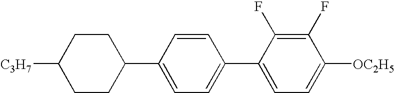 | 34 | 32 | 29 | 30 | 25 | 25 |
| 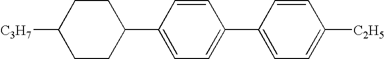 | | | 10 | 10 | 9 | 9 |
| 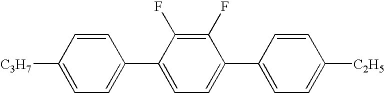 | | | 38 | 38 | 33 | 34 |
| Δn | 0.144 | 0.151 | 0.168 | 0.181 | 0.177 | 0.178 |
| TNI (° C.) | 93 | 101 | 95 | 121 | 110 | 121 |
| Δε | −5.2 | −5.0 | −4.8 | −4.0 | −4.6 | −6.2 |

As shown in Table 2, the liquid crystalline composition of the present invention has a relatively high optical anisotropy of 0.1 or greater, a relatively high nematic-isotropic transition temperature, and a significantly high negative dielectric anisotropy. Therefore, a display comprising the liquid crystalline compound of the present invention can show a high response speed in the VA mode, and can display good image quality.

Although the preferred embodiments of the present invention have been disclosed for illustrative purposes, it will be appreciated that various modifications, additions and substitutions are possible, without departing from the spirit and scope of the invention.

1. A liquid crystalline compound represented by Formula 1 below: 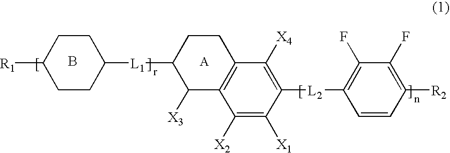
wherein R₁ and R₂ are each independently C₁₋₂₀ alkyl, cycloalkyl, alkoxy or alkenyl group in which 1-4 of hydrogen atoms may be substituted with fluorine atoms; X₁, X₂, X₃ and X₄ are each independently a hydrogen atom, F, Cl, Br, NCS, CN, CH₃, CF₃, CHF₂, CH₂F, OCF₃, OCHF₂ or OCH₂F; L₁ and L₂ are each independently a single bond, C₁₋₄ alkylene group, C₂₋₄ divalent unsaturated hydrocarbon group containing at least one double or triple bond, —COO—, —OCO—, —CH₂O—, —CF₂O—, —OCF₂—, —OCH₂—, —NHCH₂—, —CH₂NH—, —CH₂CO—, —COCH₂—, —N═N— or —NON—; n is an integer of 1 or 2; r is an integer of 0 to 2; 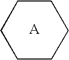
constituting the naphthalene group is a cyclohexane, cyclohexene or benzene ring; 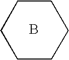
is 1,4-cyclohexylene, 1,4-phenylene or cyclohexene-1,4-diyl group in which at least one hydrogen atom may be substituted with a fluorine atom.

2. The liquid crystalline compound according to claim 1, wherein the liquid crystalline compound is one of the compounds represented by Formulae 2 to 4 below:

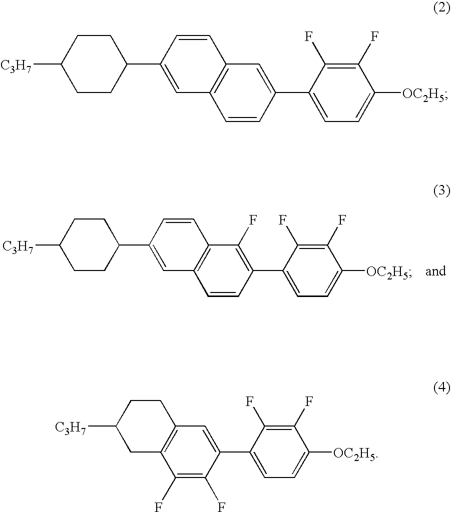

3. A liquid crystalline composition comprising the liquid crystalline compounds according to claim 1.

4. The liquid crystalline composition according to claim 3, wherein the content of the liquid crystalline compound is 3˜50% by weight, based on the total weight of the composition.

5. The liquid crystalline composition according to claim 3, wherein the liquid crystalline composition has a optical anisotropy of 0.05˜0.3.

6. The liquid crystalline composition according to claim 3, wherein the liquid crystalline composition has a dielectric anisotropy of less than −2.0.

7. The liquid crystalline composition according to claim 5, wherein the liquid crystalline composition has a optical anisotropy of 0.15˜0.25.

8. A liquid crystalline display comprising the liquid crystalline composition according to claim 3.

9. The liquid crystalline display according to claim 8, wherein the liquid crystalline display is a TFT-LCD or LcoS (Liquid Crystal on Silicon) using a vertical alignment (VA) mode.
